(19) United States
(12) Patent Application Publication
(10) Pub. No.: US 2015/0374702 A1
(43) Pub. Date: Dec. 31, 2015

(54) ANTICANCER ADJUVANT CONTAINING PENTOXIFYLLINE

(71) Applicant: CATHOLIC UNIVERSITY INDUSTRY ACADEMIC COOPERATION FOUNDATION, Seoul (KR)

(72) Inventors: Hyo Jeong KUH, Seoul (KR); Jung Ho KIM, Bucheon-si Gyeonggi-do (KR)

(73) Assignee: CATHOLIC UNIVERSITY INDUSTRY ACADEMIC CORPORATION FOUNDATION, Seoul (KR)

(21) Appl. No.: 14/769,056

(22) PCT Filed: Oct. 4, 2013
(86) PCT No.: PCT/KR2013/008900

§ 371 Date: Aug. 19, 2015

(30) Foreign Application Priority Data
Feb. 22, 2013 (KR) 10-2013-0019207

(51) Int. Cl. A61K 31/522; A61K 9/127; A61K 31/704; A61K 45/06; A61K 31/7068

(52) U.S. Cl. CPC A61K 31/522; A61K 45/06; A61K 31/7068; A61K 31/704; A61K 9/127

(57) ABSTRACT

The present invention provides an anticancer adjuvant containing pentoxifylline. Pentoxifylline inhibits a collagen synthesis in tumors and consequently increases the distribution of an anticancer drug or sensitivity of cancer cells, which thus provides an improved anticancer treatment effect.

### TECHNICAL FIELD

The present invention relates to an anticancer adjuvant containing pentoxifylline.

### BACKGROUND ART

Pentoxifylline is a drug which improves the deformability of damaged erythrocytes, suppresses thrombocyte aggregation, and improves blood circulation of blood flow damaged parts by lowering the viscosity of blood to improve the fluidity of blood. Pentoxifylline is disclosed in U.S. Pat. No. 3,422,107, and has been widely used for treating circulatory disturbances (ischemia and cerebral arteriosclerosis such as apoplexy, vertigo, headaches, and amnesia), circulatory disturbances in the blood flow of the eyes, marginal arterial circulatory disturbances (intermittent claudication, sharp pain at rest, diabetic angiopathy, atrophy, and vasoneuropathy), and the like.

Pentoxifylline is known to have activity as an inhibitor of phosphodiesterase (PDE; see the document [Meskini, N et al, Biochem. Pharm. 1994, 47(5): 781-788]) as well as activity against other biological targets. Pentoxifylline is also known to enhance blood flow properties through hemorheologic effects which lower blood viscosity and enhance erythrocyte flexibility. Pentoxifylline also increases leukocyte deformability and inhibits neutrophil adhesion and activity (see FDA label for pentoxifylline at http://www.fda.gov/cder/foi/nda/99/74962_Pentoxifylline_prntlbl.pdf). In addition to enhancing hemorheologic properties, pentoxifylline is also believed to have anti-inflammatory and anti-fibrotic properties.

Meanwhile, the extracellular matrix of tumors in the treatment of tumors is known to block the access of anticancer agents and the like, and thus, be responsible for reducing therapeutic effects of anticancer agents. This extracellular matrix is composed mainly of collagen which is a connective tissue. Further, images of tumor transplanted in mice, which are produced by using an imaging technique which is the secondary-harmonic generation (SHG), support the fact that the number of collagens is associated with the permeability of tumor. Therefore, in order to increase the therapeutic effects of the anticancer agent, it is necessary to increase the penetration of the anticancer agent into tumor cells by suppressing the synthesis of collagen in the tumor.

However, it has not been known yet that pentoxifylline suppresses the synthesis of collagen in the tumor to enhance the therapeutic effects of anticancer agents.

### DISCLOSURE

### Technical Problem

The present invention has been made in an effort to provide enhanced anticancer therapeutic effects by using the effects of inhibiting the synthesis of collagen in the tumor by pentoxifylline to use pentoxifylline and the anticancer agent in combination.

### Technical Solution

An exemplary embodiment of the present invention provides an anticancer adjuvant containing pentoxifylline.

In an exemplary embodiment of the present invention, the anticancer adjuvant may be an anticancer adjuvant in which pentoxifylline and an anticancer agent are simultaneously or sequentially administered.

In an exemplary embodiment of the present invention, the anticancer agent may be one or more selected from the group consisting of metabolic antagonists, alkylating agents, topoisomerase antagonists, microtubule antagonists, anticancer antibiotics, plant-derived alkaloids, antibody anticancer agents, and molecular targeted anticancer agents, and for example, the anticancer agent may be one or more selected from the group consisting of nitrogen mustard, imatinib, oxaliplatin, rituximab, erlotinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate, gemcitabine, doxifluridine, pemetrexed, tegafur, capecitabine, gimeracil, oteracil, azacitidine, methotrexate, uracil, cytarabine, fluorouracil, fludarabine, enocitabine, decitabine, mercaptopurine, thioguanine, cladribine, carmo fur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, lomustine, and carmustine.

In an exemplary embodiment of the present invention, the anticancer agent may be a nanoparticle preparation, for example, a liposome preparation.

In an exemplary embodiment of the present invention, for the anticancer adjuvant, pentoxifylline may be first administered, and then, the anticancer agent may be administered.

In an exemplary embodiment of the present invention, the cancer may be pancreatic cancer, liver cancer, breast cancer, lung cancer, stomach cancer, rectal cancer, gallbladder cancer, ovarian cancer, bladder cancer, colorectal cancer, lymphoma, brain cancer, uterine cancer, prostate cancer, or malignant melanoma.

### Advantageous Effects

According to the present invention, pentoxifylline and an anticancer agent are used in combination to inhibit the synthesis of collagen in the tumor and increase the distribution of an anticancer agent or the sensitivity of cancer cells, thereby enhancing the therapeutic effects of the anticancer agent.

### DESCRIPTION OF DRAWINGS

FIG. 1A illustrates a result confirming effects of the reduction in tumor size during the treatment with pentoxifylline (PTX), gemcitabine (GEM), and pentoxifylline+gemcitabine (PTX+GEM) in a mouse model, and FIG. 1B illustrates a result confirming the change in body weight in each case.

FIG. 2A illustrates a treatment schedule for pentoxifylline (PTX), saline, doxorubicin (DOX), and liposomal doxorubicin (LP-DOX). FIGS. 2B and 2C illustrate the results confirming effects of suppressing the growth of tumor after pre-treatment with pentoxifylline for 2 weeks, followed by treatment with doxorubicin or with liposomal doxorubicin, respectively.

FIG. 3A illustrates the results confirming that the distribution (Red fluorescent area) of doxorubicin and liposomal doxorubicin has been increased in the tumor tissue after treatment with pentoxifylline. C or M in the drawing indicates central or marginal regions of tumor sections. FIG. 3B is a graph showing the percent distribution areas of doxorubicin, liposomal doxorubicin, and blood vessel (CD31).

FIGS. 4A and 4B illustrate results confirming that collagen (arrow) has been reduced in mouse tumor tissues after treatment with pentoxifylline, and FIG. 4C illustrates results confirming that the mRNA expression of collagen has been reduced.

FIGS. 5A and 5B illustrate results confirming that the expression of fibrosis associated factors (α-SMA, CTGF) present in mouse tumor tissue has been reduced after treatment with pentoxifylline and that the mRNA expression of these factors has been reduced, respectively, and FIG. 5C illustrates an ELISA result confirming the levels of active TGF-β1 and total TGF-β1.

FIG. 6 is a schematic view illustrating effects of pentoxifylline on fibrosis associated factors, i.e., α-SMA, CTGF, and TGF-β.

### BEST MODE

Hereinafter, the configuration of the present invention will be described in detail.

The present invention provides an anticancer adjuvant containing pentoxifylline, a use of pentoxifylline as an anticancer adjuvant, and a method for treating cancer, including administering an anticancer agent and pentoxifylline to a subject.

Pentoxifylline is a compound having a molecular weight of 278.31 and represented by a formula of C₁₃H₁₈N₄O₃, and the general name is 1-(5-oxohexyl)-3,7-dimethylxanthine. Pentoxifylline is a methylxanthine derivative and has a structure similar to caffeine.

Collagen present in tumor reduces penetration and efficacy of an anticancer agent, and it was found that pentoxifylline increases the distribution of an anticancer agent in tumor cells or the sensitivity of cancer cells by reducing the expression of fibrosis associated factors (a-smooth muscle actin (α-SMA), connective tissue growth factor (CTGF), and the like) to inhibit the synthesis of collagen in the tumor. Therefore, when pentoxifylline is used as an anticancer adjuvant, efficacy of the anticancer agent may be enhanced.

The anticancer adjuvant containing pentoxifylline according to the present invention may be prepared by using an adjuvant which is pharmaceutically suitable and physiologically acceptable, and a solubilizing agent such as an excipient, a disintegrant, a sweetening agent, a binder, a coating agent, a leavening agent, a lubricant, a glidant or a flavoring agent may be used as the adjuvant, but the adjuvant is not limited thereto.

The anticancer adjuvant of the present invention may be formulated by including one or more pharmaceutically acceptable carriers in addition to an active ingredient for administration. Examples of carriers, excipients or diluents which may be included in the anticancer adjuvant of the present invention include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, or mineral oil, but are not limited thereto.

The anticancer adjuvants of the present invention may be a preparation for oral or parenteral administration. For example, the oral preparations may be a capsule, a tablet, a coated tablet, a slow-releasing tablet, granules, powder, syrup, a suspension, an emulsion, sap, an aerosol, and a suppository, and the parenteral preparation may be a sterilized aqueous solution, a non-aqueous solvent, a suspension, an emulsion, and a lyophilized preparation. The parenteral preparations may be administered in a typical method through an intravenous, intra-arterial, intraperitoneal, intramuscular, intrasternal, topical, rectal, or intradermal route.

In the case of the preparation for oral administration, examples of the pharmaceutically acceptable carriers include a diluent, a preservative, a binder, a lubricant, a disintegrant, a swelling agent, a filler, a stabilizer, and a combination thereof, but are not limited thereto. Carriers may also include all the components of a coating composition which may include a plasticizer, a coloring matter, a colorant, a stabilizer, and a flow agent. Examples of suitable coating materials include cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers, acrylic acid copolymers, methacrylic resins, zein, shellac, and polysaccharides, but are not limited thereto. Additionally, the coating materials may contain a typical carrier such as a plasticizer, a pigment, a colorant, a flow agent, a stabilizer, a pore former, and a surfactant. Optional pharmaceutically acceptable excipients include a diluent, a binder, a lubricant, a disintegrant, a colorant, a stabilizer, or a surfactant, but are not limited thereto.

Diluents are generally necessary to increase the volume of a solid dosage form, so that a particle size is provided for compression of tablets or formation of beads and granules. Suitable diluents include dicalcium phosphate dihydrate, calcium sulfate, lactose, sucrose, mannitol, sorbitol, cellulose, microcrystalline cellulose, kaolin, sodium chloride, dry starch, hydrolyzed starch, pregelatinized starch, silicone dioxide, titanium oxide, magnesium aluminum silicate or powdered sugar, but are not limited thereto. Binders are used to impart cohesive properties to a solid dosage formulation, and thus ensure that a tablet or bead or granule remains intact even after the composition of the dosage forms. Suitable binder materials include starch, pregelatinized starch, gelatin, sugars (including sucrose, glucose, dextrose, lactose and sorbitol), polyethylene glycol, waxes, natural and synthetic gums such as acacia, tragacanth, and sodium alginate, cellulose including hydroxypropylmethylcellulose, hydroxypropylcellulose, ethyl cellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone, but are not limited thereto.

Lubricants are used to facilitate tablet preparation. Examples of suitable lubricants include magnesium stearate, calcium stearate, stearic acid, glycerol behenate, polyethylene glycol, talc, and mineral oil, but are not limited thereto.

Disintegrants are used to facilitate disintegration or breakup of the dosage form after administration, and generally include starch, sodium starch glycolate, sodium carboxymethyl starch, sodium carboxymethylcellulose, hydraxypropyl cellulose, pregelatinized starch, clays, cellulose, alginine, gums or cross linked polymers, such as cross-linked PVP, but are not limited thereto.

Stabilizers are used to inhibit or retard drug decomposition reactions which include, for example, oxidative reactions. Suitable stabilizers include antioxidants, butylated hydroxytoluene (BHT), ascorbic acid, and salts and esters thereof; vitamin E, tocopherol and salts thereof; sulfites such as sodium metabisulphite; cysteine and derivatives thereof; citric acid; propyl gallate; and butylated hydroxyanisole (BHA), but are not limited thereto.

Oral dosage formulations, such as capsules, tablets, solutions, and suspensions, may be formulated for controlled release. For example, one or more compounds and optional one or more additional active components may be formulated into nanoparticles, microparticles, and combinations thereof, and encapsulated in a soft or hard gelatin or non-gelatin capsule or dispersed in a dispersing medium to form an oral suspension or syrup. The particles may be formed of the drug and a controlled release polymer or matrix. Alternatively, the drug particles may be coated with one or more controlled release coating agents prior to incorporation into a finished dosage form.

The preparation for parenteral administration may be prepared as an aqueous composition using a technology publicly known to the person skilled in the art. Generally, such compositions may be prepared as injectable formulations, for example, solutions or suspensions; solid forms such as micro or nanoparticles, suitable for use to prepare solutions or suspensions upon the addition of a reconstitution medium prior to injection; emulsions, such as water-in-oil (w/o) emulsions or oil-in-water (o/w) emulsions, and microemulsions thereof, liposomes, or emulsomes.

The carrier may be a solvent or dispersion medium containing, for example, water, ethanol, one or more polyols (for example: glycerol, propylene glycol, and liquid polyethylene glycol), oils (for example: vegetable oils (for example: peanut oil, corn oil, sesame oil, and the like), and combinations thereof, but is not limited thereto. The suitable fluidity may be maintained by using a coating material, such as lecithin, by maintaining the required particle size in the case of dispersion, or by using a surfactant. In addition, it is possible to include an isotonic agent sugars or salts (for example: sodium chloride), but the isotonic agent is not limited thereto.

Solutions or dispersions of the active compounds as a free acid, a free base or pharmaceutically acceptable salts may be prepared in water or another solvent or dispersing medium suitably mixed with one or more pharmaceutically acceptable excipients Examples of the excipients include surfactants, dispersants, emulsifiers, pH modifying agents, and combinations thereof, but are not limited thereto.

Suitable surfactants may be anionic, cationic, amphoteric or nonionic surface active agents. Suitable anionic surfactants include those containing carboxylate, sulfonate and sulfate ions, but are not limited thereto. Examples of anionic surfactants include sodium, potassium, and ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include quaternary ammonium compounds such as benzalkonium chloride, benzethonium chloride, cetrimonium bromide, stearyl dimethylbenzyl ammonium chloride, polyoxyethylene and coconut amine, but are not limited thereto. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-alanine, sodium N-lauryl-iminodipropionate, myristoamphoacetate, lauryl betaine, and lauryl sulfobetaine, but are not limited thereto.

Furthermore, the preparation may contain a preservative to suppress the growth of microorganisms. Suitable preservatives include paraben, chlorobutanol, phenol, sorbic acid, and thimerosal, but are not limited thereto. The formulation may also contain an antioxidant which may prevent degradation of the active ingredient(s), but is not limited thereto.

In an exemplary embodiment of the present invention, pentoxifylline and an anticancer agent may be simultaneously administered or sequentially administered. Herein, the administration includes both oral administration of tablets, capsules, and the like and parenteral administration.

In an exemplary embodiment of the present invention, the anticancer agent may be a metabolic antagonist, an alkylating agent, a topoisomerase antagonist, a microtubule antagonist, an anticancer antibiotic, a plant-derived alkaloid, an antibody anticancer agent, or a molecular targeted anticancer agent. The molecular targeted anticancer agent refers to an anticancer agent which suppresses the growth of cancer by specifically reacting with a target material such as protein to kill cancer cells or block angiogenesis. The molecular targeted anticancer agent includes imatinib, erlotinib, gefitinib, sunitinib, sorafenib, or dasatinib, and the like.

In an exemplary embodiment of the present invention, the anticancer agent may be one or more selected from the group consisting of nitrogen mustard, imatinib, oxaliplatin, rituximab, erlotinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate, gemcitabine, doxifluridine, pemetrexed, tegafur, capecitabine, gimeracil, oteracil, azacitidine, methotrexate, uracil, cytarabine, fluorouracil, fludarabine, enocitabine, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, lomustine and carmustine, but is not limited thereto.

In an exemplary embodiment, the anticancer agent may be a nanoparticle preparation. By preparing the anticancer agent as nanoparticles, the leakage of drug may be reduced, the storage stability may be enhanced, and high drug loading efficiency may be obtained. The use of a nanoparticle anticancer agent in the present invention is for enhancing the effects or stability of the anticancer, and the form of the nanoparticles is not critical. For example, the anticancer agent may be a nanoliposome preparation, but is not limited thereto. Liposome is a microscopic vesicle consisting of a phospholipid bilayer which is capable of encapsulating an active drug, and may facilitate the delivery of the active drug by enclosing the active drug in liposome. Methods of preparing nanoparticles and liposome are well known in the art (Liposome Technology 2^{nd} Edition in G. Gregoriadis, CRC Press Inc., Boca Raton (1993)).

In an exemplary embodiment of the present invention, the anticancer adjuvant may be an anticancer adjuvant in which pentoxifylline is first administered, and then, the anticancer agent is administered. The efficacy of an anticancer agent may be enhanced by subjecting pentoxifylline to pre-treatment, and then administering the anticancer agent.

The cancer, from which enhanced antitumor effects may be expected by using the anticancer adjuvant containing pentoxifylline according to the present invention, includes all the cancers in which fibroblasts are involved. The fibroblast is a cell which is important in the connective tissue, and refers to a cell which synthesizes tissue components such as collagen. Examples of the cancer in which fibroblast are involved include pancreatic cancer, liver cancer, breast cancer, lung cancer, stomach cancer, rectal cancer, gallbladder cancer, ovarian cancer, bladder cancer, colorectal cancer, lymphoma, brain cancer, uterine cancer, prostate cancer, or malignant melanoma, but are not limited thereto. Pentoxifylline may be present in the tumor to inhibit the synthesis of collagen which prevents the access of an anticancer agent, thereby increasing the distribution of the anticancer agent or the sensitivity of cancer cells.

Hereinafter, the present invention will be described in detail through the Examples. However, the following Examples are only for exemplifying the present invention, and the scope of the present invention is not limited to the following Examples.

### EXAMPLES

### Cell Lines and Reagents

A human pancreatic cancer cell line, Capan-1 was purchased from the Korean Cell Line Bank (Seoul, Korea). Under the humidity condition and 5% (v/v) CO₂ atmosphere at 37° C., cells were incubated in a RPMI-1640 medium (Gibco BRL, Grand Island, N.Y.) supplemented with 100 ug/mL of streptomycin, 100 unit/mL of penicillin (Sigma Aldrich, St. Louis, Mo.), and heat-inactivated fetal bovine serum (Welgene, Seoul, Korea). A pentoxifylline powder was purchased from Handok Pharmaceuticals Co., Ltd. (Seoul, Korea). Pentoxifylline was dissolved at a concentration of 10 mg/mL in sterilized pyrogen-free saline solution, and then the resulting solution was allowed to pass through a 0.2-μm-pore filter (Corning Costar, Germany). Doxorubicin was purchased from Ildong Pharmaceutical Co., Ltd. (Seoul, Korea), and liposomal doxorubicin was prepared as described in Enhanced Circulation Time and Antitumor Activity of Doxorubicin by Comb-like Polymer-Incorporated Liposomes. Hee Dong Han, 2007, Journal of Controlled Release. Gemcitabine was purchased from Boryung Pharmaceutical Co., Ltd. (Seoul, Korea).

Tumor Model

Female BALB/c nu/nu mice (5 to 6 weeks old) were purchased from Orient Bio Inc. (Seongnam City, Gyeonggi-do, Korea), and were raised in a germ-free animal breeding facility at the Catholic University of Korea, Catholic Medical Center (Seoul, Korea). The animals were raised in a standard state, and supplied with food and water ad libitum. The animals were taken care of in accordance with the guidelines of the Institutional Animal Care and Use Committee (IACUC) of School of Medicine of the Catholic University of Korea (Approval Number; CUMC-2010-0141-01). For induction of tumor, Capan-1 cells were harvested after the trypsin treatment, 5×10⁶ living cells were subcutaneously injected into the right flanks of the mice. After tumor was induced, the mice were sacrificed, tumor samples were obtained, and experiments were performed according to each Example to be described below.

### Example 1

### Effects of Combinatory Treatment of Pentoxifylline and Gemcitabine on Suppressing Growth of Tumor

As an effect of pentoxifylline as an anticancer adjuvant, effects of suppressing the growth of tumor by gemcitabine were experimented with or without pentoxifylline. When the size of tumor reached 200 mm³, anti-tumor effects were evaluated in the administration of saline, pentoxifylline, gemcitabine, or combination of pentoxifylline and gemcitabine. For 28 days, pentoxifylline was intraperitoneally administered at 100 mg/kg/d, and gemcitabine was intraperitoneally administered at 50 mg/kg/d twice a week. The tumor size (mm³) was calculated by using the equation: V=(a×b²)/2 (a is the largest diameter of tumor, and b is the smallest diameter of tumor). The relative tumor size was normalized with respect to the volume when a drug treatment of Capan-1 xenograft tumor was initiated. At least three mice per group were used, and the results were presented as an average standard error (SE). The statistical significance was determined by using a 2-sample t-test inferred from the Microsoft Excel 2007. All the same applies to the following Examples.

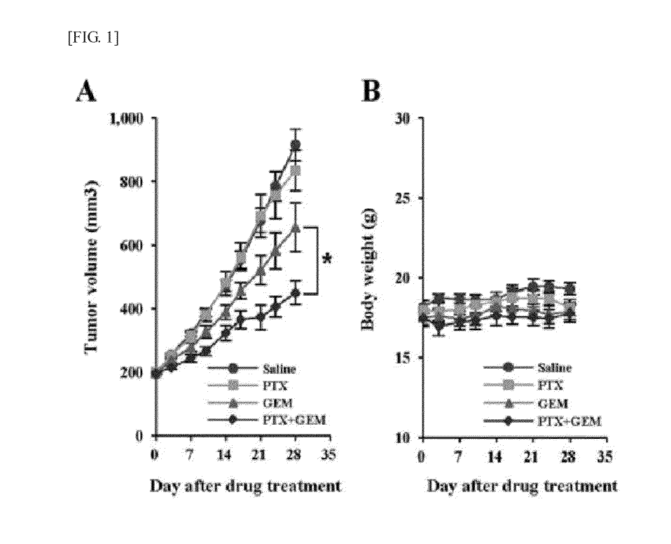

As illustrated in FIG. 1A, the tumor sizes in the combinatory treatment group as well as in the control group treated with saline solution were significantly decreased compared to those in the treatment group of gemcitabine or pentoxifylline alone (P, 0.001). After 10 days, a significant difference in the growth of tumor between the treatment group of gemcitabine alone and the combinatory treatment group appeared, and the difference was gradually increased. In the treatment group of pentoxifylline alone, anti-tumor effects did not appear compared to the control group of saline treatment. In all the groups, no significant changes in body weights were observed (FIG. 1B).

### Example 2

### Enhanced Effects of Pentoxifylline Pretreatment and Distribution of Doxorubicin and Liposomal Doxorubicin in Tumor

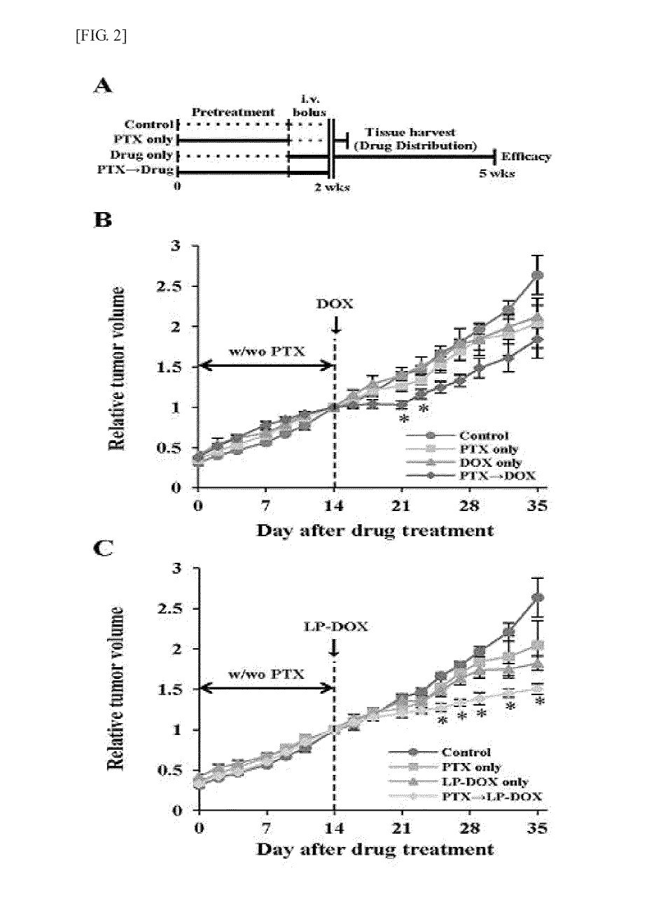

In order to confirm the mechanism of the anticancer synergistic effects of pentoxifylline, changes in the distribution and efficacy of doxorubicin were measured by performing pentoxifylline pretreatment. In order to evaluate the effects of suppressing the growth of tumor (FIG. 2A), mice were treated when the size of tumor reached 100 to 150 mm³. For two weeks, pentoxifylline pretreatment was performed at 100 mg/kg/d, and then doxorubicin or liposomal doxorubicin was intravenously injected at 8 mg/kg, and for three weeks, the tumor size was measured. The pentoxifylline pretreatment for two weeks enhanced the efficacy of doxorubicin and liposomal doxorubicin (FIG. 2). For the mice treated with pentoxifylline alone, no relative change in tumor size was observed compared to the mice treated with saline solution. However, the relative tumor size in the mice which were treated with doxorubicin after the pentoxifylline pretreatment was significantly smaller than that in the mice treated with doxorubicin alone (FIG. 2B). At day 23, the relative tumor sizes for the group treated with doxorubicin alone and the combinatory treatment group were 1.52 and 1.16, respectively (P<0.05). Likewise, after 25 days, the effects of liposomal doxorubicin on suppressing the growth of tumor had been significantly enhanced in the pentoxifylline pretreatment group (FIG. 2C). At day 35, the relative tumor sizes for the group treated with liposomal doxorubicin alone and the combinatory treatment group were 1.82 and 1.5, respectively (P<0.05). In the case of liposomal doxorubicin given with pentoxifylline pretreatment, anticancer effects were maintained for a longer period than doxorubicin given with pentoxifylline pretreatment.

In order to confirm the distribution of doxorubicin or liposomal doxorubicin in tumor, immunofluorescence was performed. Optimal cutting temperature compound (OCT)-embedded tumor samples were cut into 10 μm thick sections, the doxorubicin or liposomal doxorubicin autofluorescence was immediately observed at a wavelength of Ex/Em=540/580 nm by using a 100 W HBO mercury light source under an inverted microscope (Axiovert 200M, Carl Zeiss), imaged by an EC Plan-Neofluar 5×/0.55 NA lens, and captured by an AxioCamMR3 camera. All images were captured in 16-bit signal depth and subsequently pseudo-colored. And then, sections were fixed in cold acetone for 10 minutes, washed with PBS, and then nonspecific binding was blocked with PBS containing 5% normal goat serum for 60 minutes. Primary antibodies of a rabbit anti-collagen type I (Abcam) and a rat anti-CD31 (BD PharMingen) were incubated at 1:200 and 1:100 in a dilution buffer, respectively at 4° C. in a humidified chamber overnight. After primary antibodies were incubated, sections were washed with PBS and stained with an Alexa-488-conjugated goat anti-rabbit IgG 1:200 (Invitrogen) or a Cy3-conjugated goat anti-rat IgG 1:100 (Jackson ImmunoResearch Laboratories). And then, sections were washed with PBS, fixed, and observed under an inverted microscope (Axiovert 200M, Carl Zeiss) under the same condition as those during the measurement of the doxorubicin or liposomal doxorubicin autofluorescence. In order to combine these, CD31 staining images were taken in the portion which was the same as doxorubicin or liposomal doxorubicin. CD31 staining images were overlaid with images of doxorubicin or liposomal doxorubicin using an image overlay process of Media Cybernetics Image Pro PLUS (version 5.0). The percentage area measurement of doxorubicin or liposomal doxorubicin was performed using image analysis software OPTIMAS version 6.5 (Media Cybernetics, Silver Spring, Md.). The minimum signal level below the threshold was set based on the average level of background measured from regions which were not stained according to each tissue section.

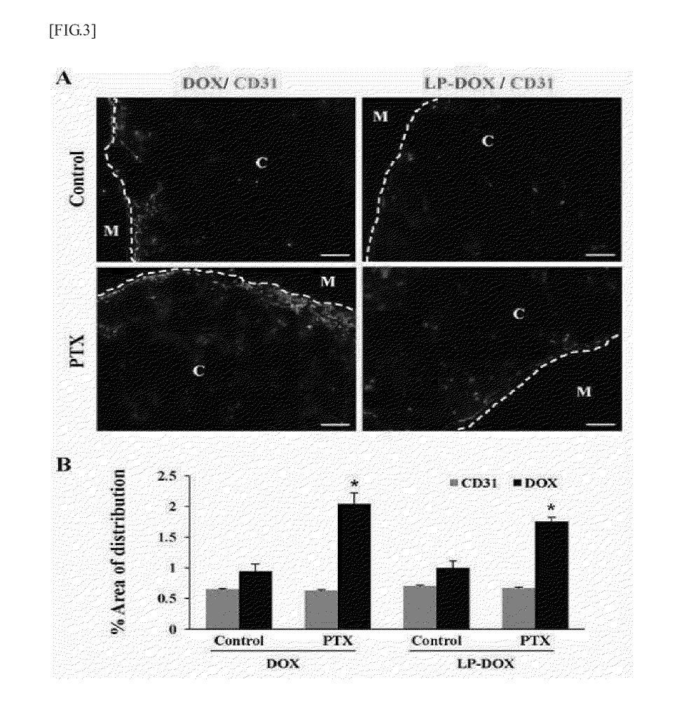

CD31 (blood vessel marker) immunostaining of doxorubicin or liposomal doxorubicin in the tumor section showed that for the pentoxifylline treatment group, the drug distribution in a portion far from the blood vessel had been enhanced (FIG. 3A). Pentoxifylline increased the area percentage of doxorubicin and liposomal doxorubicin in the tumor section by 2.17- and 1.75-folds, respectively (P<0.001) (FIG. 3B). Since the blood vessel density exhibited by CD31 immunostaining showed no difference between groups, enhanced drug distribution was not attributed to increased blood vessel concentration (FIG. 3B).

### Example 3

### Effects of Pentoxifylline on Decrease in Collagen Type I Content in Tumor

Based on the fact that tumor stroma is strongly associated with the drug distribution in tumor, the changes in collagen type I content and mRNA expression level in tumor tissues were confirmed in order to see whether the effects of pentoxifylline on improving the distribution of the anticancer agent are due to reduction in collagen type I in tumor tissue.

When the tumor diameter became 4 to 6 mm, the mice were sacrificed in order to evaluate effects of pentoxifylline on the distribution of collagen type I in tumor. For two weeks, 50 mg/kg/d of pentoxifylline and 100 mg/kg/d of pentoxifylline or saline were intraperitoneally injected into the mice. For biochemical analysis, excised tumor pieces were snap-frozen and stored at −70° C. or fixed overnight in a 10% formalin solution.

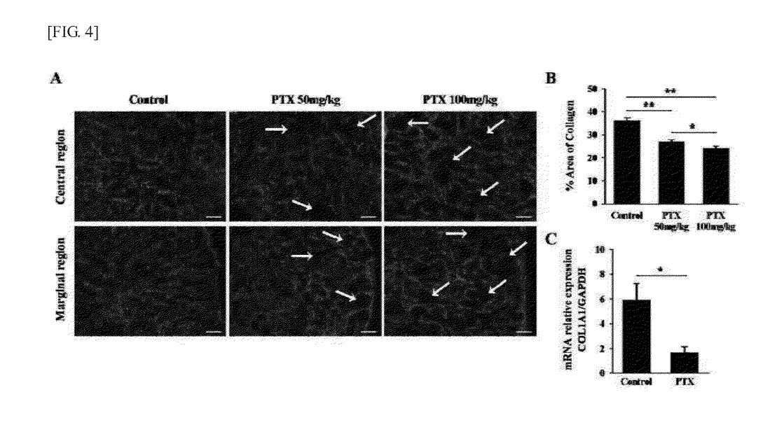

For two weeks, collagen type I contents were reduced in all the central and marginal regions of the tumor sections in the group treated with pentoxifylline (FIGS. 4A and 4B). There was a significant difference with or without pentoxifylline pretreatment. The collagen content was slightly lower in the group treated with pentoxifylline at a high dose (100 mg/kg) than in the group treated at a low dose (50 mg/kg) (P<0.05).

In order to confirm the mRNA expression level of collagen type I, RT-PCR was performed. For the quantitative real-time RT-PCR analysis, total RNAs were isolated from tumor homogenates using the Trizol method and quantified by Nanodrop spectrophotometer (ND-1000). After 1 μg of total RNAs was reverse transcribed from the tissue with AccuPower CycleScript RT PreMix (Bioneer), a quantitative real-time polymerase chain reaction was performed using the LightCycler 480 Real-Time PCR System II (Roche) with SYBR Green Master mix (Roche) according to the manufacturer's recommendation. Primers for mouse COLIA1 (collagen type I, alpha 1) were as follows.

SEQ ID No: 1:
5′-GAGCGGAGAGTACTGGATCG-3′
(mouse COLIA1 forward primer)
SEQ ID No: 2:
5′-TACTCGAACGGGAATCCATC-3′
(mouse COLIA1 reverse primer)

As a result, when compared to the control group, the mRNA expression of collagen I in the pentoxifylline pretreatment group was reduced by 72% (P<0.02) for two weeks (FIG. 4C).

### Example 4

### Effects of Pentoxifylline on Suppressing Expression of Profibrotic Growth Factors in Tumor

In order to confirm the mechanism of effects of reduction in the synthesis of collagen by pentoxifylline pretreatment and target cells, immunohistochemistry was performed so as to experiment with the expression of the profibrotic growth factors, i.e., transforming growth factor beta (TGF-β), alpha-smooth muscle actin (α-SMA), and connective tissue growth factor (CTGF).

Immunohistochemical staining for α-SMA, TGF-β1, and CTGF was performed using Dako Envision Detection System (K5007, DAKO). Paraffin-embedded, formalin fixed samples were cut into sections having a thickness of 3 μm, deparaffinized and rehydrated. For antigen retrieval, a pressure cooker of a microwave method was used as a target retrieval solution (S2375, DAKO) for 5 minutes (pH 9.0), and cooled at room temperature for 20 minutes. The samples were washed with TBS, and then nonspecific binding was blocked in PBS containing 10% normal goat serum for 60 minutes. Sections were then incubated with primary antibodies against α-SMA 1:200 (Abcam), TGF-β1 1:200 (Abcam), and CTGF 1:400 (Abcam) in a dilution buffer overnight at 4° C. in a humidified chamber. And then, sections were washed with TBS in order to block endogenous peroxidase activity and was incubated in DW containing 0.3% H₂O₂ at room temperature for 15 minutes, and then visualized by Dako Envision Detection System. After being washed with tap water, the slides were counterstained and fixed with hematoxylin, and observed under a microscope (AX70, TR-6A02, Olympus). For statistical analysis, random images were obtained at 40× magnification to contain 95% of the total area of each slide. The % area measurement of α-SMA, TGF-β1 and CTGF staining was performed using image analysis software OPTIMAS version 6.5 (Media Cybernetics, Silver Spring, Md.). The minimum signal level below the threshold was set based on the average level of background measured from regions which were not stained according to each tissue section.

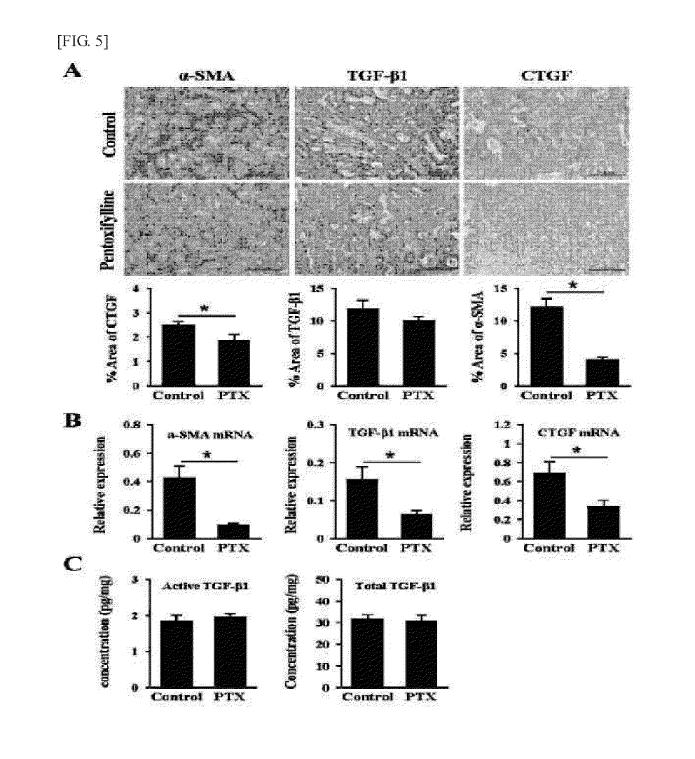

The group treated with pentoxifylline for two weeks reduced immunostaining of α-SMA and CTGF in the tumor sections by 66% (p<0.001) and 25% (p<0.05), respectively (FIG. 5A).

For the quantitative real-time RT-PCR analysis, total RNAs were isolated from tumor homogenates using the Trizol method and quantified by Nanodrop spectrophotometer (ND-1000). After 1 μg of total RNAs was reverse transcribed from the tissue with AccuPower CycleScript RT PreMix (Bioneer), a quantitative real-time polymerase chain reaction was performed using the LightCycler 480 Real-Time PCR System II (Roche) with SYBR Green Master mix (Roche) according to the manufacturer's recommendation. Primers for mouse GAPDH, human GAPDH, mouse α-SMA, mouse TGF-β1, and mouse CTGF were as follows:

SEQ ID No: 3:
5′-TGCTGAGTATGTCGTGGAGTCTA-3′
(mouse GAPDH forward primer)
SEQ ID No: 4:
5′-AGTGGGAGTTGCTGTTGAAGTC-3′
(mouse GAPDH reverse primer)
SEQ ID No:
5′-CCACCCATGGCAAATTCCATGGCA-3′
(human GAPDH forward primer)
SEQ ID No: 6:
5′-TCTAGACGGCAGGTCAGGTCCACC-3′
(human GAPDH reverse primer)
SEQ ID No: 7:
5′-ACTGGGACGACATGGAAAAG-3′
(mouse α-SMA forward primer)
SEQ ID No: 8:
5′-CATCTCCAGAGTCCAGCACA-3′
(mouse α-SMA reverse primer)
SEQ ID No: 9:
5′-CAACAATTCCTGGCGTTACCTTGG-3′
(mouse TGF-β1 forward primer)
SEQ ID No: 10:
5′-GAAAGCCCTGTATTCCGTCTCCTT-3′
(mouse TGF-β1 reverse primer)
SEQ ID No: 11:
5′-TCCCGAGAAGGGTCAAGCT-3′
(mouse CTGF forward primer)
SEQ ID No: 12:
5′-TCCTTGGGCTCGTCACACA-3′
(mouse CTGF reverse primer)

All expression data were normalized using the GAPDH expression. The relative gene expression was quantified using the ΔΔCt method (2-ΔΔCt). The quantitative real-time RT-PCR data showed that the pentoxifylline treatment for two weeks reduced the mRNA relative expression of α-SMA and CTGF by 77% and 50% (P<0.05), respectively (FIG. 5B).

The effects of pentoxifylline on the expression of TGF-β1 were confirmed through ELISA assay in addition to immunohistochemical staining.

Frozen tumors were cut into small pieces, and homogenized using Precellys 24 homogenizer (Bertin Technologies) in Tissue Extraction Reagent I (Invitrogen) containing protease inhibitor cocktail (Complete Mini, Roche). The tumor homogenate was centrifuged, and supernatant was separated and stored at −70° C. until being used for ELISA analysis. Total protein concentrations were determined by BCA protein assay kit (Pierce). Total and active TGF-β1 concentrations were determined using TGF-β1 ELISA kit (eBioscience) according to the manufacturer's protocol. Immunohistochemical results and ELISA data showed that pentoxifylline had no effects on the active TGF-β1 and the total TGF-β1 level in the tumor tissue (FIGS. 5A and 5C), but there was a big difference in mRNA expression (FIG. 5B).

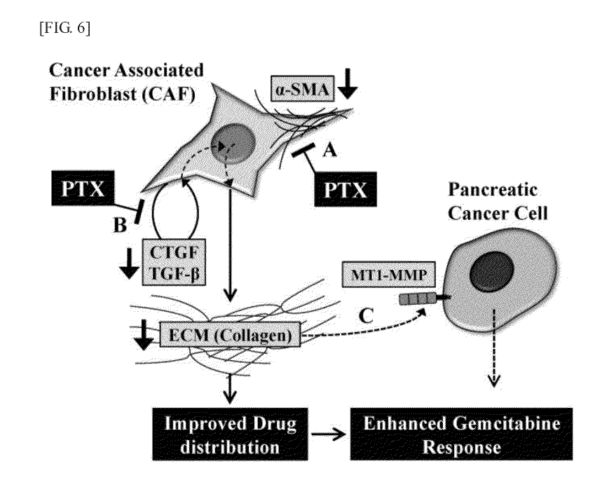

By synthesizing the above results, effects of pentoxifylline on fibrosis associated factors, i.e., α-SMA, CTGF, and TGF-β were illustrated as a schematic view (FIG. 6). As illustrated in FIG. 6, our data demonstrated that pentoxifylline reduced the expression of α-SMA to suppress the proliferation or recruitment of cancer associated fibroblasts (CAFs) (A) and suppressed the expression of CTGF and TGF-β (B) in the tumor tissue.

1. An anticancer adjuvant containing pentoxifylline.

2. The anticancer adjuvant of claim 1, wherein in the anticancer adjuvant, pentoxifylline and an anticancer agent are simultaneously or sequentially administered.

3. The anticancer adjuvant of claim 2, wherein the anticancer agent is one or more selected from the group consisting of metabolic antagonists, alkylating agents, topoisomerase antagonists, microtubule antagonists, anticancer antibiotics, plant-derived alkaloids, antibody anticancer agents, and molecular targeted anticancer agents.

4. The anticancer adjuvant of claim 3, wherein the anticancer agent is one or more selected from the group consisting of nitrogen mustard, imatinib, oxaliplatin, rituximab, erlotinib, trastuzumab, gefitinib, bortezomib, sunitinib, carboplatin, sorafenib, bevacizumab, cisplatin, cetuximab, Viscum album, asparaginase, tretinoin, hydroxycarbamide, dasatinib, estramustine, gemtuzumab ozogamicin, ibritumomab tiuxetan, heptaplatin, methyl aminolevulinic acid, amsacrine, alemtuzumab, procarbazine, alprostadil, holmium nitrate, gemcitabine, doxifluridine, pemetrexed, tegafur, capecitabine, gimeracil, oteracil, azacitidine, methotrexate, uracil, cytarabine, fluorouracil, fludarabine, enocitabine, decitabine, mercaptopurine, thioguanine, cladribine, carmofur, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, peplomycin, temozolomide, busulfan, ifosfamide, cyclophosphamide, melphalan, altretamine, dacarbazine, thiotepa, nimustine, chlorambucil, mitolactol, lomustine and carmustine.

5. The anticancer adjuvant of claim 2, wherein the anticancer agent is a nanoparticle preparation.

6. The anticancer adjuvant of claim 5, wherein the anticancer agent is a liposome preparation.

7. The anticancer adjuvant of claim 2, wherein for the anticancer adjuvant, pentoxifylline is first administered, and then, the anticancer agent is administered.

8. The anticancer adjuvant of claim 1, wherein the cancer is pancreatic cancer, liver cancer, breast cancer, lung cancer, stomach cancer, rectal cancer, gallbladder cancer, ovarian cancer, bladder cancer, colon cancer, lymphoma, brain cancer, uterine cancer, prostate cancer, or malignant melanoma.


SEQUENCE LISTING (12 sequences)

SEQ ID NO: 1 - DNA, 20 bp, Artificial sequence
  mouse COLIA1 forward primer
gagcggagagtactggatcg

SEQ ID NO: 2 - DNA, 20 bp, Artificial Sequence
  mouse COLIA1 reverse primer
tactcgaacgggaatccatc

SEQ ID NO: 3 - DNA, 23 bp, Artificial Sequence
  mouse GAPDH forward primer
tgctgagtatgtcgtggagtcta

SEQ ID NO: 4 - DNA, 22 bp, Artificial Sequence
  mouse GAPDH reverse primer
agtgggagttgctgttgaagtc

SEQ ID NO: 5 - DNA, 24 bp, Artificial Sequence
  human GAPDH forward primer
ccacccatggcaaattccatggca

SEQ ID NO: 6 - DNA, 24 bp, Artificial Sequence
  human GAPDH reverse primer
tctagacggcaggtcaggtccacc

SEQ ID NO: 7 - DNA, 20 bp, Artificial Sequence
  mouse alpha-SMA forward primer
actgggacgacatggaaaag

SEQ ID NO: 8 - DNA, 20 bp, Artificial Sequence
  mouse alpha-SMA reverse primer
catctccagagtccagcaca

SEQ ID NO: 9 - DNA, 24 bp, Artificial Sequence
  mouse TGF-alpha1 forward primer
caacaattcctggcgttaccttgg

SEQ ID NO: 10 - DNA, 24 bp, Artificial Sequence
  mouse TGF-alpha1 reverse primer
gaaagccctgtattccgtctcctt

SEQ ID NO: 11 - DNA, 19 bp, Artificial Sequence
  mouse CTGF forward primer
tcccgagaagggtcaagct

SEQ ID NO: 12 - DNA, 19 bp, Artificial Sequence
  mouse CTGF reverse primer
tccttgggctcgtcacaca